(19) United States
(12) Patent Application Publication
(10) Pub. No.: US 2025/0276157 A1
(43) Pub. Date: Sep. 4, 2025

(54) GUIDEWIRE CLAMPING DEVICE AND COLLET

(71) Applicant: Merit Medical Systems, Inc., South Jordan, UT (US)

(72) Inventors: Richard P. Jenkins, Bluffdale, UT (US); Michael Dean Haslam, Sandy, UT (US); Richard D. Jenkins, Kennett Square, PA (US)

(21) Appl. No.: 19/066,721

(22) Filed: Feb. 28, 2025

Related U.S. Application Data
(60) Provisional application No. 63/560,315, filed on Mar. 1, 2024.

(51) Int. Cl. A61M 25/09

(52) U.S. Cl. CPC A61M 25/09041; A61M 2025/09125

(57) ABSTRACT

A guidewire clamping device system that includes a handle with a body that defines a lumen that extends from a proximal end of the handle to a distal end of the handle. The distal end of the handle comprising a coupling member. The system further includes a collar that defines a lumen that extends from a proximal end to a distal end of the collar. The collar is configured to couple to the distal end of the handle. The system further includes a collet that includes a body with a tubular section with a lumen, and a plurality of ramps that are flared outward from the tubular section in an unconstrained state. The collet is disposed within a lumen of the handle and the collar. The collar is coupled to the handle which biases the plurality of ramps of the collet inward to a constrained state.

### RELATED APPLICATIONS

This application claims priority to U.S. Provisional Application No. 63/560,315, filed on Mar. 1, 2024, and titled, “Guidewire Clamping Device and Collet,” which is hereby incorporated by reference in its entirety.

### TECHNICAL FIELD

The present disclosure relates generally to the field of guidewire clamping devices. More particularly, some embodiments relate to a collet used in guidewire clamping devices to clamp and secure a guidewire.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a hemostasis torque assembly utilizing a guidewire clamping device according to one embodiment of the present disclosure.

FIG. 2A illustrates a guidewire clamping device according to one embodiment of the present disclosure.

FIG. 2B illustrates the guidewire clamping device of FIG. 2A clamping a guidewire.

FIG. 3 illustrates an exploded view of a guidewire clamping device according to one embodiment of the present disclosure.

FIG. 4A illustrates a perspective view of a handle of a guidewire clamping device according to one embodiment of the present disclosure.

FIG. 4B illustrates another perspective view of the handle of FIG. 4A.

FIG. 5A illustrates a perspective view of a collar of a guidewire clamping device according to one embodiment of the present disclosure.

FIG. 5B illustrates another perspective view of the collar of FIG. 5A.

FIG. 6A illustrates a perspective view of a collet of a guidewire clamping device according to one embodiment of the present disclosure.

FIG. 6B illustrates a side view of the collet of FIG. 6A.

FIG. 6C illustrates an end view of the collet of FIG. 6A.

FIG. 6D illustrates another perspective view of the collet of FIG. 6A.

FIG. 7A illustrates a cross-sectional view of a guidewire clamping device with a collet in an unconstrained configuration, according to one embodiment of the present disclosure.

FIG. 7B illustrates a cross-sectional view of the guidewire clamping device of FIG. 7A with the collet in a partially constrained configuration.

FIG. 7C illustrates a cross-sectional view of the guidewire clamping device of FIG. 7A with the collect in a constrained configuration.

### DETAILED DESCRIPTION

This disclosure describes a guidewire clamping device for securing a guidewire. The guidewire clamping device may be used in conjunction with another medical device, such as a hemostasis valve. In some instances, when access to a cardiovascular system of a patient is sought, the guidewire clamping device helps provide control to the practitioner of inserted guidewires, such as rotating the guidewire to control the position of a distal tip of the guidewire. Various medical procedures require manipulation of the guidewire. In some procedures, guidewires are designed to navigate blood vessels to reach a target vessel segment. Once at the target vessel segment, the guidewire may act as a guide for catheters or other devices. Thus, the position of a guidewire may further facilitate proper placement of medical devices. In some procedures, a distal tip of the guidewire must be placed in the desired position at the target vessel segment to properly place a medical tool.

In some operations, after a medical device is guided to a treatment site, the guidewire is removed while the medical device is used. A physician may need to later reinsert the guidewire to guide additional medical tools to the treatment site. However, reinsertion of the guidewire may alter the axial displacement and/or rotational angle of the guidewire.

Further, the positional relationship between the end of a guidewire and the end of a catheter may also be related to storage and shipment of a catheter system. For example, to assist with insertion into a blood vessel, a catheter may have a curved end. For shipment, the catheter may be coupled to the hemostasis valve and a guidewire inserted into the catheter. However, if the guidewire extends to the curved end of the catheter, the guidewire may exert a force on the catheter that causes the curved end to lose its form. Thus, in some instances, a guidewire may be placed offset from the curved end during shipping and storage.

The guidewire clamping device system that works in conjunction with other medical devices may include a handle with a gripping member. The practitioner is able to grip the handle of the guidewire clamping device because the handle is bigger than the guidewire. The gripping member may have a textured surface to increase friction for better gripping. The ability of the practitioner to grip the handle enables the practitioner to rotate the guidewire when the guidewire clamping device clamps the guidewire.

The guidewire clamping device system may further include a collar with a mating mechanism configured to selectively couple to the handle. A collet within the collar may selectively provide a clamping force to maintain a position of the guidewire extending through the collet. The handle may couple to the collar and selectively alter the clamping force of the collet based on the axial position of the handle relative to the collar. The guidewire may be rotated by the practitioner by rotating the handle when the guidewire clamping device clamps the guidewire. When the guidewire clamping device does not clamp the guidewire, the practitioner may advance and retract the guidewire thought a lumen of the guidewire clamping device.

The phrase “coupled to” is broad enough to refer to any suitable coupling or other form of interaction between two or more entities, including mechanical, fluidic, and thermal interaction. Thus, two components may be coupled to each other even though they are not in direct contact with each other. The phrase “attached to” refers to interaction between two or more entities which are in direct contact with each other and/or are separated from each other only by a fastener of any suitable variety (e.g., mounting hardware or an adhesive).

Axial displacement refers to movement in a longitudinal direction of an elongated member. Rotational displacement refers to a change in angle of an elongated member about a longitudinal axis.

The terms “proximal” and “distal” are opposite directional terms. The distal end of a device or component is the end of the component that is furthest from the physician during ordinary use. The proximal end refers to the opposite end, or the end nearest the physician during ordinary use.

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

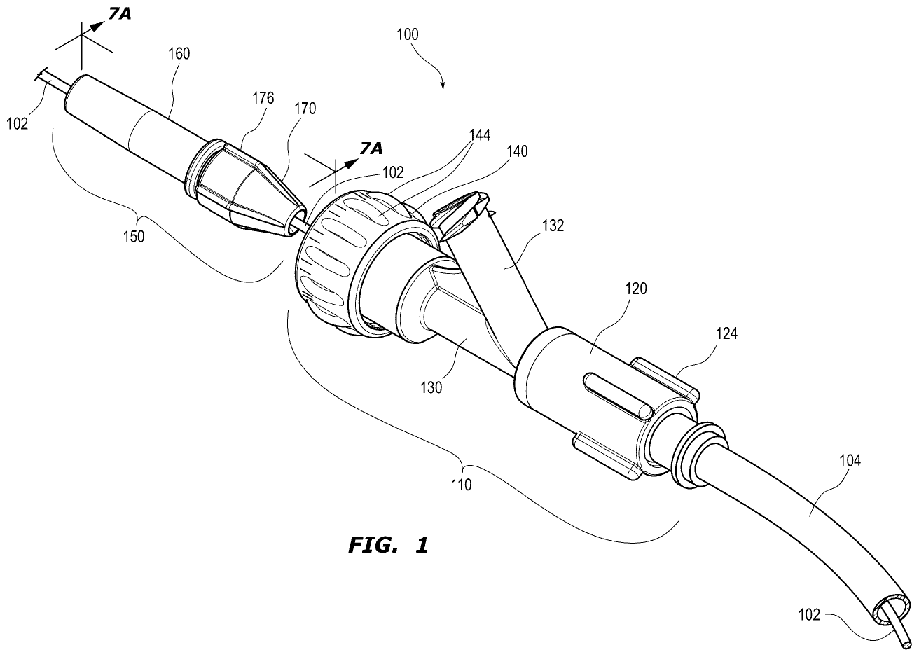

FIG. 1 illustrates a perspective view of a hemostasis torque assembly 100 according to one embodiment of the present disclosure. The hemostasis torque assembly 100 may facilitate manipulation of a guidewire 102 while limiting fluid loss. The hemostasis torque assembly 100 illustrated in FIG. 1 includes a guidewire clamping device 150 and a hemostasis valve 110. The guidewire clamping device 150 enables a practitioner to control and manipulate an inserted guidewire. In some embodiments, the guidewire clamping device 150 and the hemostasis torque assembly 100 may be selectively coupled to each other. In some embodiments, the guidewire clamping device 150 may be used in connection with the hemostasis valve 110, but those components may only interact via other components (e.g., a guidewire) with no direct attachment or interaction between the hemostasis valve and the guidewire clamping device 150. Still further, as discussed above, the guidewire clamping device 150 may be used in conjunction with a variety of different medical devices and the present disclosure is not limited to using the guidewire clamping device 150 with the hemostasis valve 110.

The guidewire clamping device 150 is configured to selectively secure, engage, or grip a guidewire 102 and facilitate displacement, such as translation or rotation, of the guidewire 102 by manipulation of the guidewire clamping device 150. For example, as shown, a practitioner may decrease a diameter of an opening of the guidewire clamping device 150 through which the guidewire 102 is placed. When the diameter of the opening is equivalent to the diameter of the guidewire 102, the guidewire clamping device 150 physically engages with the guidewire 102 such that the position of the guidewire clamping device 150 relative to the guidewire 102 is fixed. Components configured for gripping or securing the guidewire 102 are discussed in more detail below.

The guidewire clamping device 150 may permit the practitioner to have enhanced or greater rotational control and axial displacement control of the guidewire 102. For instance, the guidewire clamping device 150 has a greater diameter than the guidewire 102, thereby providing a larger surface to grip while the practitioner is manipulating the guidewire 102. Also, when the guidewire clamping device 150 is secured to the guidewire 102, the coupling of the guidewire clamping device 150 and the hemostasis valve 110 (e.g., directly or via the guidewire 102) may prevent or limit axial displacement.

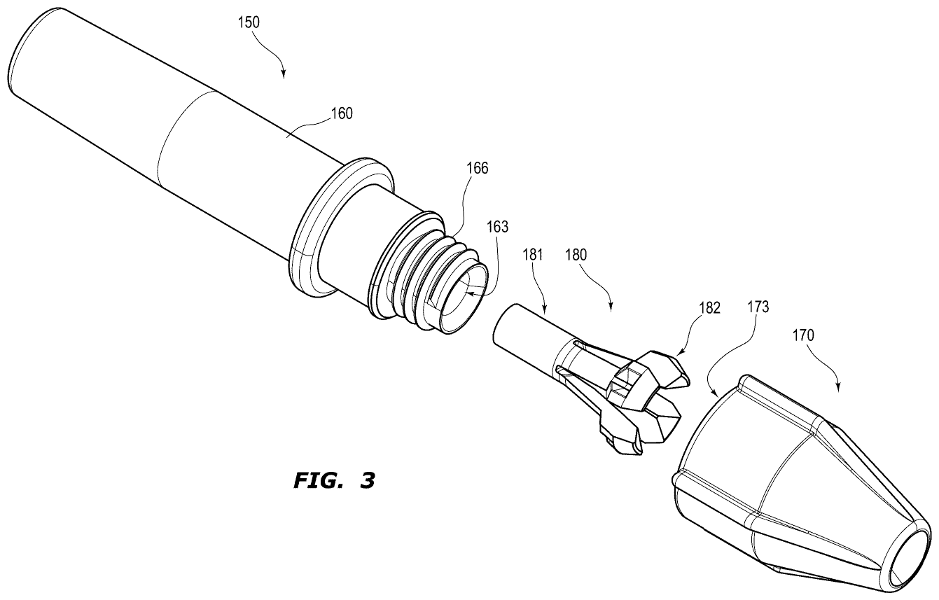

The guidewire clamping device 150 may comprise a handle 160, a collar 170, and a collet 180 (not shown in FIG. 1, see FIG. 3). The collet 180 may have a segmented portion. A diameter of the segmented portion may be adjusted by applying or removing a compressing force. The handle 160 and the collar 170 may provide the compressing force. For example, when the handle 160 is axially displaced relative to the collar 170, the compressing force may be altered. In some embodiments, the axial displacement may be controlled via a coupling member, e.g., a threaded interface between the handle 160 and the collar 170. In some embodiments, gripping elements 176 may assist in rotating the collar 170 to adjust the axial displacement between the handle 160 and the collar 170. In some embodiments, the coupling member may facilitate a direct push or pull of the handle 160 in the axial direction to alter the compression force. In some embodiments, the compressing force may be automatically controlled via an actuator.

The hemostasis valve 110 is configured to prevent fluid loss while allowing medical devices to enter a valve lumen. The hemostasis valve 110 may include an intravascular access connector 120, a body 130, and a compression nut 140.

The intravascular access connector 120 may be located at a distal end of the body 130. The intravascular access connector 120 may couple to various medical devices such as a catheter 104 or an introducer. The intravascular access connector 120 may include gripping elements 124 to assist in attaching the intravascular access connector 120 to a medical device.

The body 130 may include a valve lumen to provide a passage from a distal end to a proximal end. Medical devices, tissue samples, or fluids may be distributed through the valve lumen. A seal within the compression nut 140 may provide a hemostatic entrance to the valve lumen. A sidearm 132 may be coupled to the body 130 and provide a secondary access passage to the valve lumen.

The hemostasis torque assembly 100 may comprise a seal actuator, configured to selectively seal the valve lumen. The seal actuator may be configured to alter the diameter of a sealable opening in the seal, allowing a practitioner to obstruct fluid passage through the valve lumen. The seal actuator may facilitate sealing of the valve lumen around an elongate instrument such as the guidewire 102. In the illustrated embodiment, the compression nut 140 is an exemplary embodiment of a seal actuator. In the illustrated embodiment, a practitioner may use the compression nut 140 to adjust a seal. For example, the diameter of an opening in the seal may change based on a rotational movement of the compression nut 140. The compression nut 140 may include gripping elements 144 to assist in rotating the compression nut 140. The gripping elements (124, 144, 154) in the illustrated embodiment include a plurality of ridges. However, the gripping elements (124, 144, 154) may comprise other structures, such as divots, high-friction surfaces, and/or slots.

In some embodiments, the hemostasis valve 110 may provide resistance to prevent or limit unintentional rotational movement of an inserted guidewire 102. In some embodiments, the collar 170 and the compression nut 140 may comprise indicators that indicate the rotational position of the guidewire clamping device 150 relative to the hemostasis valve 110.

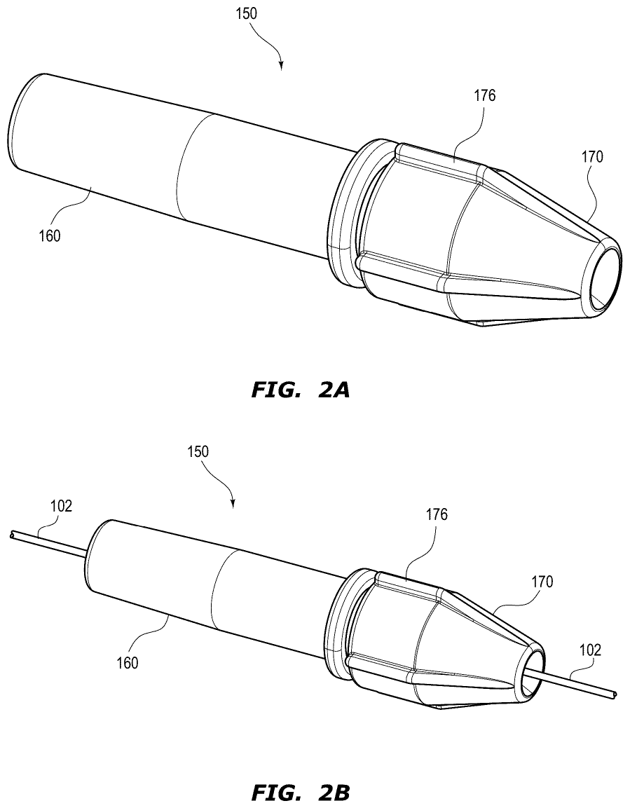

FIG. 2A illustrates a detailed perspective view of the guidewire clamping device 150 without a guidewire disposed within the guidewire clamping device 150. FIG. 2B illustrates a perspective view of the guidewire clamping device 150 with a guidewire 102 extending through the guidewire clamping device 150. The guidewire 102 may be advanced and slid through the guidewire clamping device 150 by a practitioner until the guidewire 102 reaches the target location in the patient. The guidewire clamping device 150 is also configured to clamp and secure the guidewire 102. For example, after a distal end of the guidewire 102 reaches the target location in the patient, the practitioner may twist the collar 170 in a first predetermined direction which clamps the collet 180 (not shown in FIG. 2B) onto the guidewire 102. Twisting the collar 170 in the first predetermined direction axially displaces the collar 170 relative to the handle 160 which provides the compressing force to the collet 180. The amount of twisting can affect the amount of compressive force. When the guidewire 102 is clamped and secured by the guidewire clamping device 150, the guidewire 102 cannot axially slide through the guidewire clamping device 150. When the guidewire 102 is clamped and secured by the guidewire clamping device 150, the practitioner may grip the handle 160 and rotate the guidewire clamping device 150 and simultaneously rotate the guidewire 102, which also rotates the distal tip of the guidewire 102. The practitioner may also axially adjust the position of the guidewire 102 when the guidewire 102 is clamped and secured by the guidewire clamping device 150. The practitioner may advance or retract the relative position of the guidewire clamping device 150 which simultaneously advances or retracts the axial position of the guidewire 102.

The practitioner may release the guidewire 102 from the guidewire clamping device 150 by untwisting the collar 170 in a second predetermined direction, e.g., the opposite direction of the first predetermined direction, to unclamp the guidewire 102 from the guidewire clamping device 150. Twisting the collar 170 in the second predetermined direction axially displaces the collar 170 relative to the handle 160 which causes the collet 180 to release the guidewire 102. When the guidewire 102 is released and unclamped from the guidewire clamping device 150, the practitioner may again axially advance or retract the guidewire 102 relative to the guidewire clamping device 150.

FIG. 3 illustrates an exploded view of the guidewire clamping device 150 illustrated in FIG. 1. The guidewire clamping device 150 includes the handle 160, the collar 170, and the collet 180. The handle 160 may couple to the collar 170 via the coupling member, such as a threaded interface 166. The collet 180 may be partially disposed within both the handle 160 and the collar 170. For example, a distal end 182 of the collet 180 may be housed within a lumen 173 the collar 170, and a proximal end 181 of the collet 180 may be housed within a lumen 163 the handle 160. In the illustrated embodiment, the distal end 182 of the collet 180 has a bulbous end that does not fit within the lumen 163 of the handle 160 but does fit within the lumen 173 of the collar 170. Because the collet 180 does not entirely fit within the lumen 163 of the handle 160 the bulbous distal end 182 never enters the lumen 163. However, the lumen 173 of the collar is designed to accommodate the bulbous distal end 182 in the unconstrained configuration. As the collar 170 and the handle 160 couple together, the bulbous distal end is axially displaced toward the handle 160 without the bulbous distal end 182 ever entering the lumen 163 of the handle 160 due to its size.

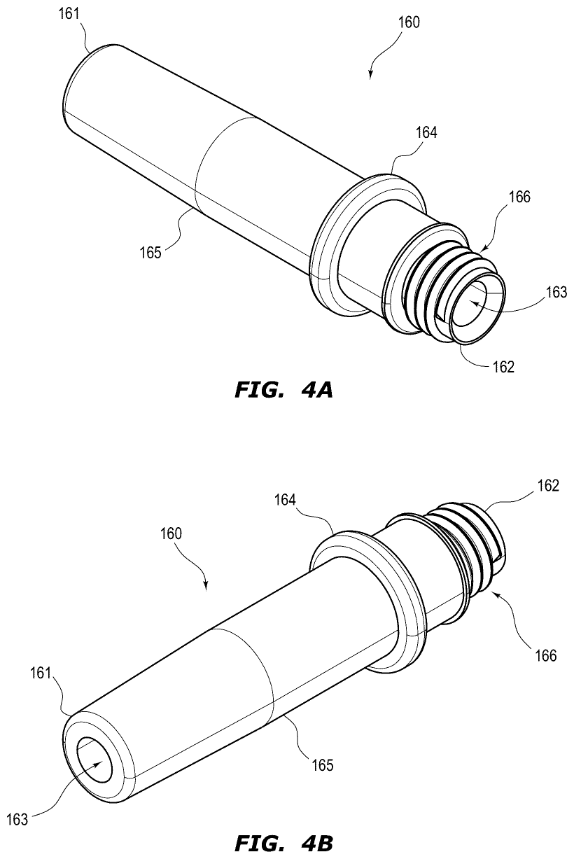

FIGS. 4A and 4B illustrate perspective views of the handle 160. The handle 160 includes a proximal end 161 and a distal end 162 with the lumen 163 extending from the proximal end 161 to the distal end 162 of the handle 160. FIG. 4A illustrates a distal end perspective view and FIG. 4B illustrates a proximal end perspective view. The distal end 162 of the lumen 163 is configured to receive the proximal end 181 of the collet 180.

The handle 160 comprises a flange 164 disposed between the proximal end 161 and the distal end 162. The proximal side of the flange 164 includes a gripping member 165 that may be gripped by the practitioner. The gripping member 165 may comprise a textured surface or a rough surface. The textured or rough surface increases the friction of the gripping member 165, thus increasing the ability of the practitioner to grip the gripping member 165. The distal end 162 of the handle 160 comprises the threaded interface 166 that is engageable with the collar 170.

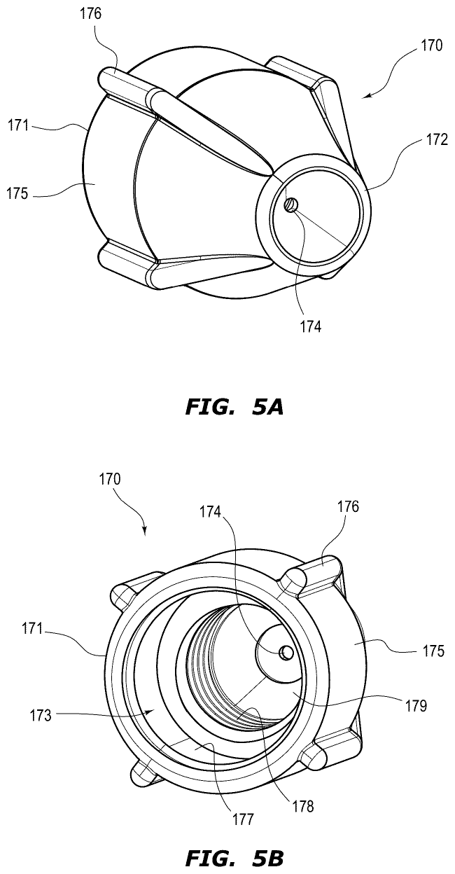

FIGS. 5A and 5B illustrate perspective views of the collar 170. The collar 170 includes a proximal end 171 and a distal end 172 with the lumen 173 extending from the proximal end 171 to the distal end 172 of the collar 170. The lumen 173 decreases in diameter from the proximal end 171 to the distal end 172. FIG. 5A illustrates a distal end perspective view and FIG. 5B illustrates a proximal end perspective view. The proximal end 171 of the collar 170 is configured to receive the distal end 182 of the collet 180. The distal end 172 of the lumen 173 includes an aperture 174. The aperture 174 is sized to receive different sizes of guidewires commercially available, specifically up to a diameter of 0.046 inches.

An outer surface 175 of the collar 170 includes the plurality of gripping elements 176. The plurality of gripping elements 176 helps the practitioner to grip the collar 170 in order to rotate the collar 170 to either clamp the guidewire 102 of release the guidewire 102. To clamp the guidewire 102, the practitioner rotates the collar 170 in the first predetermined direction and to release the guidewire 102, the practitioner rotates the collar 170 in the second predetermined direction, e.g., the opposite direction. In some embodiments, the first predetermined direction is clockwise and the second predetermined direction is counterclockwise.

FIG. 5B illustrates the proximal end perspective view. An inner surface 177 of the collar 170 includes a threaded interface 178 that engages with the threaded interface 166 of the handle 160 and a distal inner surface 179 that is configured to engage with an outer surface 194 of the collet 180, as discussed below.

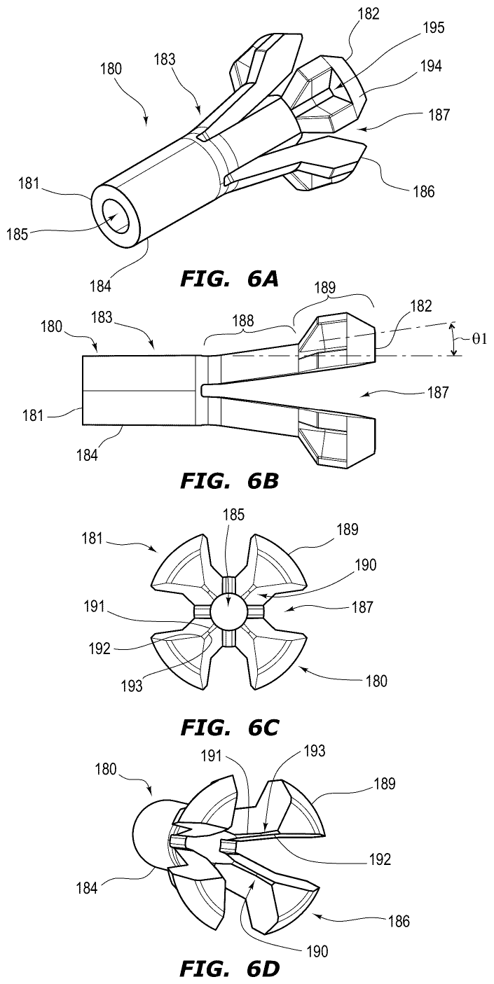

FIGS. 6A-6D illustrate different views of the collet 180. FIG. 6A illustrates a proximal perspective view of the collet 180. FIG. 6B illustrates a side view of the collet 180. FIG. 6C illustrates a distal end view of the collet 180. FIG. 6D illustrates a distal end perspective view of the collet 180.

As discussed previously, the collet 180 comprises a body 183 with the proximal end 181 and the distal end 182. The proximal end 181 of the body 183 includes a tubular section 184 with a lumen 185. The tubular section 184 of the collet 180 is insertable into the lumen 163 of the handle 160.

The tubular section 184 of the body 183 transitions into a plurality ramps 186. The plurality of ramps 186 are separated circumferentially by a plurality of gaps 187. In other words, a gap 187 is disposed between each of the adjacent ramps 186. The plurality of ramps 186 flare radially outward from the tubular section 184. In the illustrated embodiment, the collet 180 comprises four ramps 186. However, the present disclosure is not so limited, and the collet 180 may have more or less than four ramps 186.

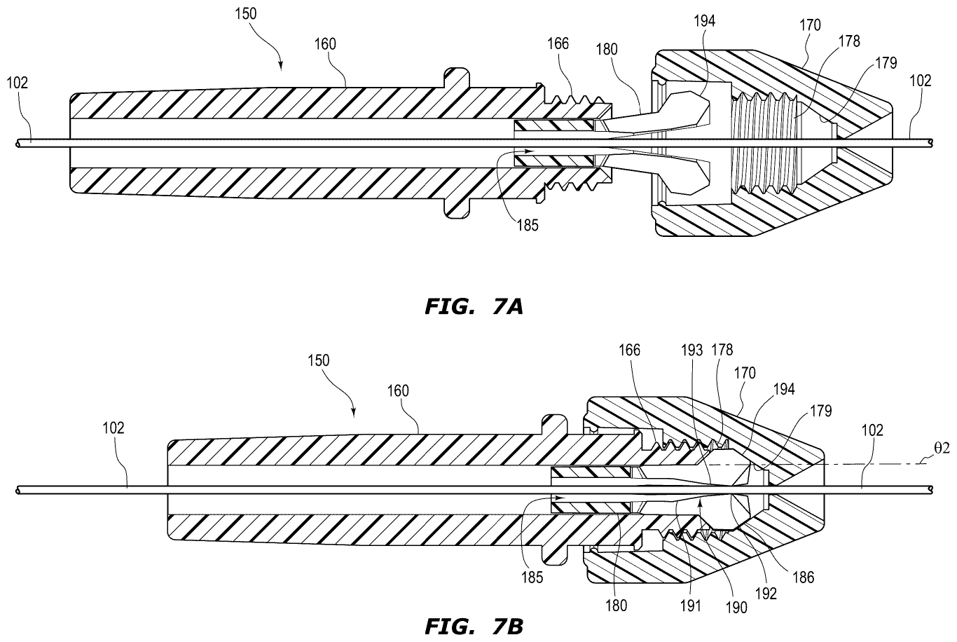

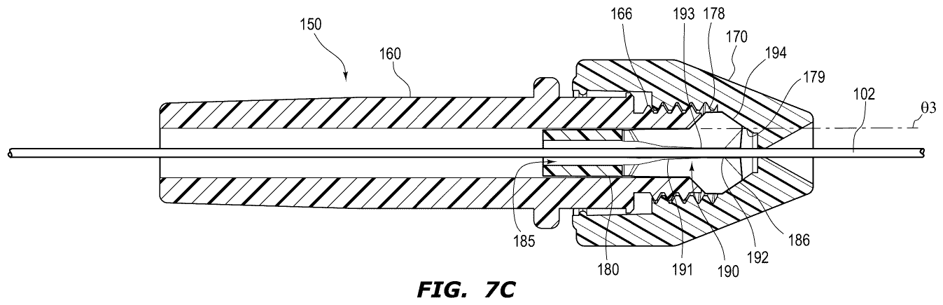

FIG. 6B illustrates a side view of the collet 180. In an unconstrained state, the plurality of ramps 186 flare radially outward from the tubular section 184 at a first predetermined angle θ1 or draft angle. The first predetermined angle θ1 may be between 2 degrees and 30 degrees. In some embodiments, the first predetermined angle θ1 may be between 5 degrees and 10 degrees. Each ramp 186 comprises an elongate portion 188 and a bulbous distal portion 189. The elongate portion 188 of each ramp 186 may be inserted into and constrained by the lumen 163 of the handle 160 and the bulbous distal portion 189 is inserted into the collar 170 such that the first predetermined angle θ1 of the ramps 186 are flattened to second predetermined angle θ2 of approximately zero degrees (see FIG. 7B). The bulbous distal portion 189 of each ramp 186 is sized so that it does not fit within the lumen 163 of the handle 160 and is configured to be disposed within the lumen 173 of the collar 170. As discussed previously, twisting of the collar 170 onto the handle 160 is configured to further constrain the collet 180 from a partially constrained state to a constrained state in which the ramps 186 are biased inward to from the second predetermined angle θ2 up to a third predetermined angle θ3. The twisting of the collar 170 is configured to secure the guidewire 102 with the collet 180. The third predetermined angle θ3 is less than second predetermined angle θ2 and the third predetermined angle θ3 may be approximately zero degrees. In some embodiments, the second predetermined angle θ2 and the third predetermined angle θ3 may be negative. The ramps 186 can be constrained anywhere between the first predetermined angle θ1 and the third predetermined angle θ3. While the ramps 186 are at least partially constrained the elongate portion 188 of the ramps 186 may slide into the lumen 164 of the handle 160.

Each of the plurality of ramps 186 comprises a contact surface 190 on an inner portion of the ramp 186. The contact surfaces 190 are illustrated in FIGS. 6C and 6D. The contact surface 190 of each ramp 186 is configured to engage with the guidewire 102 when the collet 180 transitions from the unconstrained state to the constrained state. The contact surface 190 of each ramp 186 may be flat or slightly curved, e.g., concave curve. In other words, and in contrast to designs wherein the gripping portion of a collet come to a point or apex, the contact surface 190 of the collet 180 may be configured to engage a guidewire along a surface (190) defining a width, rather than only along an apex, point, or line. A point, apex, or sharp edge of other designs could cut through or puncture coatings on some guidewires or other devices. This can be especially problematic when the collet is brass or another metal as it can more easily cut through the coating of the guidewire 102. The collet 180 of the present design comprises contact surfaces 190 that may distribute the gripping load and avoid cutting or otherwise damaging the wire surface.

As illustrated in FIGS. 6D, the contact surface 190 of each ramp 186 extends a majority of a length of each ramp 186 in a longitudinal direction of the collet 180, including the elongate portion 188 and the bulbous distal portion 189.

In some embodiments, the contact surface 190 of each ramp 186 may comprise a change in slope in the longitudinal direction of the collet 180. In the illustrated embodiment of FIGS. 6C and 6D, the contact surface 190 may comprise a first sloped surface 191, a second sloped surface 192, and a transition point 193 between the first sloped surface 191 and the second sloped surface 192. In certain circumstances, when the collet 180 is constrained, a distal portion of the second sloped surface 192 is configured to engage with the guidewire 102 before the first sloped surface 191 or the transition point 193. In other words, the second sloped surface 192 of each contact surface engages the guidewire 102 before the first sloped surface 191 or the transition point 193.

In some embodiments, when the collet 180 is constrained, the transition point 193 is configured to engage with the guidewire 102 before the first sloped surface 191 or the second sloped surface 192. In other words, the transition point 193 of each contact surface engages the guidewire 102 before the contact surface 190 on opposing sides of the transition point 193.

In some embodiments, when the collet 180 is constrained, a portion of the first sloped surface 191 is configured to engage with the guidewire 102 before the second sloped surface 192 or the transition point 193. In other words, the first sloped surface 191 of each contact surface engages the guidewire 102 before the second sloped surface 192 or the transition point 193.

In some embodiments, the collet 180 is fabricated from a compliant material, such as a plastic, not a metal or a metal alloy. The term compliant signifies that the material may compress. For example, when the contact surface 190 of the collet 180 engages with the guidewire 102 when the collet 180 is being constrained, the collet 180 may compress. The compression of the collet 180 may increase the surface area of the contact surface 190 that engages with the guidewire 102 when the collet 180 is constrained. The compression of the collet 180 also prevents excessive force being applied to the guidewire 102 and prevent the potential cutting of the coating of the guidewire 102. Exemplary compliant materials include plastics such as polyoxymethylene (e.g., Delrin®), nylon, polycarbonate, polybutylene terephthalate, acrylonitrile butadiene styrene, and the like, and any suitable combination thereof.

As discussed above, in some embodiments, the contact surface 190 includes the first sloped surface 191, the second sloped surface 192, and the transition point 193. When the collet 180 is fabricated from a compliant material, the transition point 193 is configured to engage with the guidewire 102 before the first sloped surface 191 or the second sloped surface 192. Due to the compliant nature of the collet 180, the transition point 193 will compress and then the first sloped surface 191 and the second sloped surface 192 will engage with the guidewire 102 and increase the surface area of the engagement between the contact surface 190 and the guidewire 102. In some circumstances the contact surface 190 conforms to the shape of the guidewire 102.

The guidewire clamping device 150 is configured to accommodate a variety of different sized guidewires. Specifically, the collet 180 is design to accommodate guidewires with a diameter that range between 0.009 inches and 0.046 inches. The various features of the collet 180 enable the collet 180 to accommodate a wide variety of different size diameter guidewires. Specifically, the flared design of the ramps 186 enables the collet 180 to accommodate larger diameter guidewires, up to 0.046 inches. The design of the ramps 186 of the collet 180 also enables the collet 180 to accommodate smaller diameter guidewires, down to 0.009 inches. Prior art collets require different sized collets to accommodate the same range of available guidewires, a smaller collets to accommodate smaller guidewires and larger collets to accommodate larger guidewires.

In some embodiments, the outer surface 194 of the bulbous distal portion 189 comprises a slot 195. The outer surface 194 is configured to engage with the distal inner surface 179 of the collar 170. The slot 195 disposed on the outer surface 194 of the bulbous distal portion 189 of the plurality of ramps 186 make the collet 180 light and avoids voids in the collet 180 during the molding process.

FIGS. 7A-7C illustrate the effects of an adjustment to the threaded interface 166 of the handle 160 and an adjustment to the threaded interface 178 of the collar 170.

FIG. 7A illustrates a cross-sectional view of the guidewire clamping device 150 of FIG. 1 taken through plane 7A-7A in a first position where the guidewire 102 is unsecured and may be manipulated in an axial or rotational direction. As shown, a diameter of the lumen 185 within the collet 180 may be large enough to allow the guidewire 102 to pass through. In this first position, the practitioner may push or pull the guidewire 102 freely to adjust its longitudinal position. For example, when a practitioner places the guidewire 102 in a patient, the practitioner may advance the guidewire 102 to a treatment site while the guidewire clamping device 150 is in this first position.

FIG. 7B illustrates a cross-sectional view analogous to the view of FIG. 7A of the guidewire clamping device 150 of FIG. 1 in a second position or a partially constrained state. To secure the guidewire 102, the collar 170 is coupled to the handle 160. In the illustrated embodiment, threaded interface 178 of the collar 170 engages with the threaded interface 166 of the handle 160. As the practitioner inserts the handle 160 and the collet 180 into the collar 170, the distal inner surface 179 of the collar 170 engages with the outer surface 194 of the collet 180 due to axial displacement between the collar 170 and the handle 160. This engagement forces the collet 180 into the lumen 163 of the handle 160, which biases the plurality of ramps 186 inward and constrains the collet 180. The plurality of ramps 186 are constrained from the first predetermined angle θ1 toward the second predetermined angle θ2.

In the second position, where the guidewire 102 is secured by the contact surface 190 of each of the plurality of ramps 186 of the collet 180. As illustrated in the FIG. 7B, the distal end of the second contact surface 192 engages with the guidewire 102. The engagement between the contact surface 190 of the plurality of ramps 186 of the collet 180 clamps onto the guidewire 102 and secures the guidewire 102 in place. As discussed previously, when the guidewire 102 is secured, the practitioner may rotate the handle 160, which simultaneously rotates the guidewire 102.

FIG. 7C illustrates a cross-sectional view analogous to the view of FIGS. 7A and 7B of the guidewire clamping device 150 of FIG. 1 in a third position or a fully constrained state. To further secure the guidewire 102, the threaded interface 178 of the collar 170 engages with the threaded interface 166 of the handle 160 is twisted in the first predetermined direction which causes additional axial displacement. As the practitioner twists the collar 170 in the first predetermined direction, the distal inner surface 179 of the collar 170 further engages with the outer surface 194 of the collet 180. This engagement forces the contact surface 190 of each of the plurality of ramps 186 to further engage with the guidewire 102. The collet 180 is fabricated from a compliant material. In other words, the collet 180 compresses and conforms to the shape of the guidewire 102. In the third position, the plurality of ramps 186 are constrained to the third predetermined angle θ3.

In the third position, where the guidewire 102 is secured by the contact surface 190 of each of the plurality of ramps 186 of the collet 180. In some embodiments, only the second contact surface 192 of each of the plurality of ramps 186 engages with the guidewire 102. In some embodiments, the second contact surface 192 and the transition point 193 of each of the plurality of ramps 186 engagers with the guidewire 102. In some embodiments, the entire contact surface 190 engages with the guidewire 102, including the first sloped surface 191, the second sloped surface 192, and the transition point 193. The engagement between the contact surface 190 of the plurality of ramps 186 of the collet 180 clamps onto the guidewire 102 and secures the guidewire 102 in place. As discussed previously, when the guidewire 102 is secured, the practitioner may rotate the handle 160, which simultaneously rotates the guidewire 102.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

We claim:

1. A collet for a guidewire clamping device, the collet comprising:
a body comprising:
a tubular section with a lumen;
a plurality of ramps that are flared outward from the tubular section in a unconstrained state,
wherein in a constrained state, the plurality of ramps are biased inward such than a contact surface of each of the ramps are configured to contact a guidewire disposed within the body and secure the guidewire when all of the contact surface of each ramp engages the guidewire.

2. The collet of claim 1, wherein the plurality of ramps are flared outward at a draft angle between 2 degrees and 30 degrees.

3. The collet of claim 1, wherein the contact surface of each ramp of the plurality of ramps is not a point.

4. The collet of claim 1, wherein the contact surface of each ramp of the plurality of ramps is flat.

5. The collet of claim 1, wherein the contact surface of each ramp of the plurality of ramps extends a majority of a length of the ramp in a longitudinal direction of the collet.

6. The collet of claim 1, wherein the contact surface of each ramp of the plurality of ramps comprises a change in slope in a longitudinal direction of the collet.

7. The collet of claim 1, wherein each ramp comprises a slot disposed on an outer surface of the ramp.

8. The collet of claim 1, wherein the collet comprises four ramps.

9. The collet of claim 1, wherein the collet is fabricated from a compliant material such that when the plurality of ramps clamp onto the guidewire, the compliant material is configured to compress.

10. The collet of claim 1, wherein the collet is a plastic.

11. The collet of claim 10, wherein the collet is polyoxymethylene.

12. A guidewire clamping device system comprising:
a handle comprising a body that defines a lumen that extends from a proximal end of the handle to a distal end of the handle, the distal end of the handle comprising a coupling member;
a collar that defines a lumen that extends from a proximal end to a distal end of the collar, wherein the collar is configured to couple to the distal end of the handle; and
a collet comprising:
a body comprising:
a tubular section with a lumen; and
a plurality of ramps that are flared outward from the tubular section in a unconstrained state,
wherein the collet is disposed within a lumen of the handle and the collar, and
wherein the collar is coupled to the handle, the collar biases the plurality of ramps of the collet inward to a constrained state to constrain the collet.

13. The guidewire clamping device system of claim 12, further comprising a guidewire that extends through the handle, the collet, and the collar,
wherein in the constrained state, the plurality of ramps are biased inward by the collar such than a contact surface of each of the ramps are configured to contact the guidewire and secure the guidewire when all of the contact surface of each ramp engages the guidewire.

14. The guidewire clamping device system of claim 13, wherein the guidewire comprises a diameter between 0.009 inches and 0.046 inches and the collet is configured to secure guidewires with a diameter between 0.009 inches and 0.046 inches.

15. The guidewire clamping device system of claim 13, wherein the contact surface of each ramp of the plurality of ramps comprises a change in slope in a longitudinal direction of the collet, and wherein the change in slope and the contact surface distal to the change in slope engages the guidewire.

16. The guidewire clamping device system of claim 15, wherein the collet is fabricated from a compliant plastic material, and wherein the plurality of ramps of the collet compress when the contact surfaces engage with the guidewire.

17. A method of clamping a guidewire comprising:
inserting a guidewire into a guidewire clamping device, the guidewire clamping device comprising:
a handle comprising a body that defines a lumen that extends from a proximal end of the handle to a distal end of the handle, the distal end of the handle comprising a coupling member;
a collar that defines a lumen that extends from a proximal end to a distal end of the collar, wherein the collar is configured to couple to the distal end of the handle; and
a collet comprising:
a body comprising:
a tubular section with a lumen;
a plurality of ramps that are flared outward from the tubular section in a unconstrained state;
twisting the collar of the guidewire clamping device to constrain the plurality of ramps inward such that a contact surface of each of the plurality of ramps clamp the guidewire.

18. The method of claim 17, wherein the collet is fabricated from a compliant plastic material such that twisting the collar compresses the collet and the contact surface of each ramp conforms to the guidewire.

19. The method of claim 17, wherein the contact surface of each ramp of the plurality of ramps comprises a transition point that changes a slope of the contact surface, and wherein the transition point and the contact surface distal to the transition point of each contact surface engages the guidewire.

20. The method of claim 17, wherein the collet of the guidewire clamping device is configured to secure guidewires with diameters between 0.009 inches and 0.046 inches.
